(19) United States
(12) Patent Application Publication
(10) Pub. No.: US 2012/0263663 A1
(43) Pub. Date: Oct. 18, 2012

(54) COMPOSITIONS AND METHODS FOR PREVENTION OF PHOTOAGING

(75) Inventors: Eric F. Bernstein, Wynnewood, PA (US); Yann Echelard, Jamaica Plains, MA (US); Paul Donald Forbes, Malvern, PA (US)

(73) Assignee: GTC BIOTHERAPEUTICS, INC., Framingham, MA (US)

(21) Appl. No.: 13/531,439

(22) Filed: Jun. 22, 2012

Related U.S. Application Data
(62) Division of application No. 11/060,041, filed on Feb. 17, 2005, now abandoned.
Application No. 11/060,041 is a continuation of application No. 09/913,697, filed on Jan. 28, 2002, now abandoned.
(60) Provisional application No. 60/121,118, filed on Feb. 22, 1999.

(51) Int. Cl. A61K 38/57; A61P 17/02; A61P 17/00; A61P 17/16; A61P 39/06

(52) U.S. Cl. 424/59

(57) ABSTRACT

Methods of preventing photoaging and other types of sun damage by topically applying a composition containing a serine protease inhibitor or milk are provided. Pharmaceutical compositions comprising serine protease inhibitors or milk for the prevention of photoaging and other types of sun damage are also provided.

### BACKGROUND OF THE INVENTION

The effects of ultraviolet radiation from exposure to the sun on human skin are a growing concern for today's longer-lived population. The majority of changes associated with an aged appearance result from chronic sun-damage (Warren et al., J. Am. Acad. Dermatol., 1991, 25:751-760; Frances, C. and Robert, L., Int. J. Dermatol., 1984, 23:166-179). Dramatic alterations of the superficial dermis accompany the deep wrinkles and laxity common in photoaged skin. The major histopathologic alteration of photoaged skin is the accumulation of material which, on routine histopathologic examination, has the staining characteristics of elastin and is, thus, termed solar elastosis. Immunohistochemical staining has shown the poorly-formed fibers comprising solar elastosis to be composed of elastin (Chen et al., J. Invest. Dermatol., 1986, 87:334-337; Mera et al., Br. J. Dermatol., 1987, 117:21-27) fibrillin (Chen et al., J. Invest. Dermatol., 1986, 87:334-337; Dahlback et al., J. Invest. Dermatol., 1990, 94:284-291; Bernstein et al., J. Invest. Dermatol., 1994, 103:182-186) and versican, the normal components of elastic fibers (Zimmerman et al., J. Cell. Biol., 1994, 124:817-825). A coordinate increase in elastin, fibrillin and versican mRNAs has been demonstrated in fibroblasts derived from photodamaged skin, as compared to fibroblasts derived from normal skin from the same individuals (Bernstein et al., J. Invest. Dermatol., 1994, 103:182-186). Elevated elastin mRNA levels in sun-damaged skin result from enhanced elastin promoter activity, as shown by transient transfections of fibroblasts with a DNA construct composed of the human elastin promoter linked to the chloramphenicol acetyltransferase (CAT) reporter gene (Bernstein et al., J. Invest. Dermatol., 1994, 103:182-186).

Neutrophil elastase has been suggested to be an important mediator in the development of solar elastosis resulting from continued exposure to UVB (See Abstract from Ciba-Found. Symp., 1995, 192:338-46; discussion 346-7). Using an elastase-deficient hairless mouse model and specific small molecular weight elastase inhibitors, it has been shown that attenuation of neutrophil elastase activity results in a pronounced diminuation in the severity of UVB or chemically-induced skin tumors (Starcher et al. J. Invest. Dermatol., 1996, 107:159-163).

A deficiency in alpha 1-antitrypsin has been suggested to allow proteases such as neutrophil elastase to destroy dermal elastin and, thus produce cutis laxa in Marshall's syndrome, a rare pediatric skin disease that is characterized by acquired localized neutrophilic dermatitis (Sweet's disease), followed by loss of elastic tissue in the dermis and cutis laxa (Hwang et al. Arch. Dermatol., 1995, 131(10):1175-7). Alpha 1-proteinase inhibitor, also referred to herein as alpha 1-antitrypsin, is approved by the Food and Drug Administration as a plasma product for the treatment of hereditary alpha 1-antitrypsin deficiency. Alpha 1-antitrypsin has also been disclosed for use in the treatment of atopic dermatitis (Wachter, A. M. and Lezdey, J. Annals of Allergy, 1992, 69:407-414).

Alpha 1-antitrypsin is a member of the serine protease inhibitor (serpin) supergene family. Serpins are a superfamily of inhibitors involved in the mediation of a variety of biological processes essential to survival of a host. Members of the serpin family play a role in a great number of biological processes including, but not limited to, inflammation, fertilization, tumor migration, neurotropism, and heat shock. The serpin with the highest naturally occurring plasma concentration is alpha 1-antitrypsin. This serpin has activity toward both tryptic and chymotryptic proteases.

It has now been found that topical application of serine proteases such as alpha 1-antitrypsin prevents photoaging and other skin damage resulting from exposure to solar, and more specifically, ultraviolet radiation.

### SUMMARY OF THE INVENTION

In the present invention, a new use is provided for serine proteases such as alpha 1-antitrypsin. It has now been demonstrated that topical application of alpha-1 antitrypsin protects against photoaging and other sun-damage such as sunburn and skin cancer caused by solar radiation. Accordingly, serine proteases with alpha 1-antitrypsin-like activities are believed to be useful as sunscreen agents. Compositions for use as sunscreen agents comprising serine proteases with alpha 1-antitrypsin like activities are also provided.

### DETAILED DESCRIPTION OF THE INVENTION

Profound changes take place in the superficial dermis as a result of chronic sun-exposdre. The major alteration is the deposition of massive amounts of abnormal elastic material, termed solar elastosis. It has been shown that solar elastosis is accompanied by elevations in elastin and fibrillin mRNAs and elastin promoter activity.

A transgenic mouse model which contains the human elastin promoter linked to a chloramphenicol acetyltransferase (CAT) reporter gene for testing compounds that may inhibit cutaneous photodamage has been developed. These mice express human elastin promoter activity in a tissue-specific and developmentally regulated manner. Promoter activity can be studied in this model as a function of small increases in ultraviolet radiation, demonstrating the sensitivity of the assay. In addition, quantitative data can be obtained after only a single exposure to ultraviolet radiation. A test compound is applied to the skin of a transgenic mouse capable of expressing the human elastin promoter. The transgenic mouse is then exposed to solar radiation and human elastin promoter activity in the mouse is determined. The human elastin promoter activity is then compared to that in transgenic mice also exposed to an equivalent dose of solar radiation which were not treated with the test compound to determine whether or not the test compound provided protection against the solar radiation. Since elastin promoter activation is a primary event in cutaneous aging, these mice represent a mouse model of human photoaging.

Using this transgenic mouse line, the ability of alpha 1-antitrypsin to inhibit the effects of solar radiation on human elastin promoter activity was determined. Alpha 1-antitrypsin is produced in the milk of transgenic goats. Accordingly, in these experiments, 5 mice received either no treatment, 10 mice were treated with a 20 mg/ml solution of alpha 1-antitrypsin in goat's milk applied topically to the back, and 10 mice were treated with a solution of goat's milk alone applied topically to the back. A group of mice was also treated with saline only. Approximately fifteen minutes after application of the goat's milk containing alpha 1-antitrypsin, goat's milk alone, or saline these mice were exposed to 20 human minimal erythema doses (MEDs) of solar simulating radiation (SSR). Following phototreatment, the backs of the mice were rinsed twice with 70% isopropyl alcohol pads to remove any excess alpha 1-antitrypsin. This procedure was repeated over three consecutive days.

Mice were sacrificed and skin harvested for determination of CAT activity 24 hours after the third phototreatment. The baseline CAT activity of control mice receiving neither radiation nor alpha 1-antitrypsin was standardized to a value of one. Relative increases in CAT activity were 14.4+3.1 (mean+S.D.) in mice treated with goat's milk alone and 4.5+1.0 in mice treated with goat's milk containing alpha 1-antitrypsin. Thus, topical application of the serpin alpha 1-antitrypsin produced a 69% reduction in CAT activity. In addition, it was found that milk alone provided 12% protection as compared to the saline control animals.

Accordingly, topical application of a composition comprising alpha 1-antitrypsin or other serpins with alpha 1-antitrypsin like activities to the skin provides protection against photoaging and other sun-damage such as sunburn and skin cancer. By “other serpins with alpha 1-antitrypsin-like activities”, it is meant serine protease inhibitors with similar activity toward both tryptic and chymotryptic proteases as alpha 1-antitrypsin. Such serpins include both naturally occurring serine protease inhibitors and mutants rationally engineered to have similar activities and specificity to alpha 1-antitrypsin. Methods of rationally engineering serine proteases and their inhibitors are known. See, for example, Dang et al. Nature Biotechnology, 1997, 15:146-149.

Examples of compositions comprising a serpin with alpha 1-antitrypsin like activities include, but are not limited to creams, lotions and sprays. Methods of formulating serpins into creams, lotions and sprays as well as pharmaceutical additives for such formulations are well known to those skilled in the art. As will be obvious to those skilled in the art upon this disclosure, such compositions may further comprise secondary or additional sunscreens or free radical scavengers such as, but not limited to, Vitamin C and Vitamin E and analogs thereof. In a preferred embodiment, a composition comprising a serpin is applied to the skin prior to exposure to the sun. However, application of these compositions subsequent to the exposure can also mitigate any damage resulting to the skin from this exposure. It is believed that these compositions of the present invention will be especially useful in protecting individuals with heightened sensitivities to the sun, such as, but not limited to, individuals undergoing psoralen treatment for cancer, psoriasis and other skin conditions; individuals undergoing photodynamic therapy for skin cancer, psoriasis and other skin conditions; individuals suffering from genetic repair defects such as xeroderma pigmentosa, albinism or other conditions resulting from decreased endogenous melanin pigment.

Further, as demonstrated herein topical application of a composition comprising milk or a product derived therefrom also provides protection against photoaging and other sun-damage such as sunburn and skin cancer. Accordingly, compositions such as creams, lotions and sprays which comprise milk or a product derived therefrom can also be formulated for, use in protecting against photodamage and other sun-damage in normal individuals and those with a heightened sensitivity to the sun.

The following nonlimiting examples are provided to further illustrate the present invention.

### EXAMPLES

### Example 1

### Transgenic Mice Expressing the Human Elastin Promoter

A homozygous line of transgenic mice expressing the 5.2-kb human elastin promoter linked to a CAT reporter gene was used. Hsu-Wong et al., J. Biol. Chem., 1994, 269:18072-18075. These mice express the human elastin promoter in a tissue-specific and developmentally regulated manner. Mice four or five days old were used since at this age, visible hair growth is not yet present.

### Example 2

### Solar Simulating Radiation

A Multiport Solar Simulator (Solar Light Company, Philadelphia, Pa.) containing a xenon arc lamp filtered through a Schott WG 320 filter (Schott Glaswerke, Mainz, Germany) was used to administer solar simulating radiation (SSR). The output of the solar, simulator was measured by means of a 3D UV meter (Solar Light Company) and displayed as human minimal erythema doses (MEDs). The emission spectrum of the lamp closely simulates solar radiation reaching the earth's surface. The light guides from the solar simulator were placed in light contact with the dorsal surface of the mice, which were restrained to prevent movement while SSR was administered. Unirradiated control mice were also restrained without receiving SSR.

### Example 3

### CAT Assay

To measure the expression of the human elastin promoter/CAT reporter gene construct in the skin of transgenic, mice and in fibroblast cultures established from these animals, CAT activity was determined. For extraction of the CAT from skin, the specimens were homogenized in 0.25 Tris-HCl, pH 7.5, using a tissue homogenizer (Brinkmann Instruments, Inc. Westbury, N.Y.). The homogenates were centrifuged at 10,000×g for 15 minutes at 4° C. and the protein concentration in the supernatant determined by a commercial protein assay kit (Bio-Rad Laboratories, Richmond, Calif.). Aliquots of the supernatant containing 100 μg of protein were used for assay of CAT activity by incubation with [¹⁴C] chloramphenicol in accordance with well-known procedures. The acetylated and non-acetylated forms of radioactive chloramphenicol were separated by thin-layer chromatography and CAT activity was determined by the radioactivity in the acetylated forms as a percent of the total radioactivity in each sample.

1-12. (canceled)

13. A composition for preventing photoaging and other sun-damage comprising:
a serine protease inhibitor;
at least one second sunscreen or free radical scavenger; and
at least one pharmaceutical additive.

14. The composition of claim 13, wherein the serine protease inhibitor is alpha 1-antitrypsin.

15. The composition of claim 13, wherein the composition further comprises milk or a product derived therefrom.

16. The composition of claim 15, wherein the milk or product derived therefrom is produced by a transgenic animal.

17. The composition of claim 13, wherein the at least one second sunscreen or free radical scavenger is chosen from Vitamin C, Vitamin E and analogs thereof.

18. The composition of claim 13, wherein the composition is a topical application chosen from creams, lotions and sprays.

19. A composition for preventing solar elastosis comprising:
a milk or a product derived therefrom; and
a serine protease inhibitor.

20. The composition of claim 19, further comprising at least one second sunscreen or free radical scavenger.

21. The composition of claim 20, wherein the at least one second sunscreen or free radical scavenger is chosen from Vitamin C, Vitamin E and analogs thereof.

22. The composition of claim 19, further comprising at least one pharmaceutical additive.

23. The composition of claim 19, wherein the serine protease inhibitor is present in an amount effective to protect the skin against photoaging and sunburn.

24. The composition of claim 19, wherein the serine protease inhibitor is alpha 1-antitrypsin.

25. The composition of claim 24, wherein the alpha 1-antitrypsin is present in an amount of about 20 mg/ml of solution of milk.

26. The composition of claim 19, wherein the milk or a product derived therefrom is produced by a goat and comprises recombinantly expressed alpha 1-antitrypsin.

27. The composition of claim 19, wherein the composition is a topical application chosen from creams, lotions and sprays.
